(19) United States
(12) Patent Application Publication
(10) Pub. No.: US 2012/0034635 A1
(43) Pub. Date: Feb. 9, 2012

(54) METHOD FOR AMPLIFYING ADENOSINE TRIPHOSPHATE AND METHOD AND REAGENT FOR DETECTING THE CONCENTRATION OF MICROORGANISMS

(75) Inventors: Ching-Yi Hsu, Kaohsiung City (TW); Meng-Shun Huang, Banqiao City (TW); Hwan-You Chang, Changhua County (TW); Hui-Ju Lee, Taichung County (TW); Ming-Rong Ho, Kaohsiung City (TW)

(73) Assignee: INDUSTRIAL TECHNOLOGY RESEARCH INSTITUTE, Hsinchu County (TW)

(21) Appl. No.: 12/954,824

(22) Filed: Nov. 26, 2010

(30) Foreign Application Priority Data
Aug. 6, 2010 (TW) 099126238

(51) Int. Cl. C12Q 1/66; C12P 19/32

(52) U.S. Cl. 435/8; 435/92

(57) ABSTRACT

A method for amplifying adenosine triphosphate is provided, including mixing adenosine triphosphate sulfurylase, adenosine 5′ phosphosulfate, adenylate kinase, uridine triphosphate, acetate kinase, acetyl phosphate, luciferin and luciferase in the presence of ATP to form a mixture, and reacting the mixture to amplify ATP. A method and reagent for detecting a concentration of microorganisms are also provided.

### CROSS REFERENCE TO RELATED APPLICATIONS

This application claims the benefit of priority to Taiwan Patent Application No. 09126238, filed Aug. 6, 2010. The disclosure of the application is incorporated herein by reference.

### BACKGROUND

1. Technical Field

The disclosure relates to a method for amplifying adenosine triphosphate (ATP), and in particular relates to a method for increasing the sensitivity of ATP concentration detection.

2. Description of the Related Art

For industrial uses or clinical analysis, microorganisms in water are examined in amounts usually less than a standard value or nearly zero. However, according to conventional detection techniques, precisely detecting trace amounts of microorganisms in water is currently not available. The microorganisms in water usually accumulate for reproduction and secrete polymers, such as polysaccharide, to encapsulate the microbial body forming a biofilm. It is known that it is difficult to eliminate biofilm, by cleaning chemical reagents, strong acids, strong alkali or the ozone, after being formed. As an example in Taiwan, for the high tech industry, commonly, when biofilm is discovered in a tube, the one solution is to replace the whole tube with a new one. In the medical field, antibiotics are incapable of penetrating biofilm to kill the microorganism. Therefore, detection techniques with greater sensitivity have been developed in relevant fields, to detect microorganisms at low concentrations in water before a biofilm is formed to prevent biofilm formation.

Luminescence detection is one of the conventional methods for detecting the concentration of microorganisms in water. Luminescence detection uses luciferase and luciferin reacting with adenosine triphosphate (ATP) in microbial cells to emit light. The quantity of microorganism is determined according to the emitted light detected by a luminometer. However, detection is heavily influenced by manual expertise, and sensitivity is not high, with a high cost.

WO 03/044222A1 discloses a reagent and method for simplified ATP measurement by forming glucose-6-phosphate in the presence of acetyl phosphate and glucose with acetate kinase and glucokinase or hexokinase, wherein a visible coloration reaction is induced with the combined use of glucose-6-phosphate dehydrogenase and diaphorase, thereby allowing visual determinations.

WO01/53513A1 discloses an ATP regeneration reaction system, wherein adenosine monophosphate (AMP) is converted to adenosine diphosphate (ADP) and the resultant ADP is converted back to ATP and a polyphosphoric acid compound by treatment with polyphosphoric acid synthase. Another ATP regeneration reaction system is further disclosed, wherein AMP is converted to ADP by treatment with phosphotransferase in the presence of a polyphosphoric acid compound and the resultant ADP is converted to ATP by treatment with polyphosphoric acid synthase.

WO2006/118093A1 discloses a method for analyzing adenosine triphosphate contained in a sample comprising four steps: 1. a step of mixing AMP, phosphoenolpyruvate, adenylate kinase and pyruvate kinase with the sample and incubating the mixture for a predetermined period; 2. a step of adding an acid and pyruvate oxidase to the mixture and incubating the mixture for a predetermined period; 3. a step of adding an acid iron (II) and a color developing reagent and incubating the mixture for a predetermined period; and 4. a step of determining the concentration of ATP from the color of the mixture.

Based on the limitations of conventional luminescence detection techniques, a novel method for detecting the concentration of microorganisms with enhanced sensitivity and stability is proposed.

### SUMMARY

A detailed description is given in the following embodiments with reference to the accompanying drawings.

The disclosure is based on luminescence detection of ATP and adds a further ATP regeneration technique to increase the detectable amount of ATP and, accordingly, increase the sensitivity and stability of detection for the concentration of microorganisms.

In one aspect, the disclosure provides a method for amplifying adenosine triphosphate (ATP), comprising mixing adenosine triphosphate sulfurylase (ATP-sulfurylase), adenosine 5′ phosphosulfate (APS), adenylate kinase (ADK), uridine triphosphate (UTP), acetate kinase, acetyl phosphate, luciferin and luciferase in the presence of ATP to form a mixture, and reacting the mixture to amplify ATP.

In another aspect, the disclosure provides a method for detecting the concentration of microorganisms in a sample, comprising mixing the sample with adenosine triphosphate sulfurylase (ATP-sulfurylase), adenosine 5′ phosphosulfate (APS), adenylate kinase (ADK), uridine triphosphate (UTP), acetate kinase, acetyl phosphate, luciferin and luciferase to form a mixture; reacting the mixture to produce luminescence; and determining the concentration of microorganisms according to the luminescence.

In another aspect, the disclosure provides a reagent for detecting a concentration of microorganisms, comprising adenosine triphosphate sulfurylase (ATP-sulfurylase), adenosine 5′ phosphosulfate (APS), adenylate kinase (ADK), uridine triphosphate (UTP), acetate kinase, acetyl phosphate, luciferin and luciferase.

### BRIEF DESCRIPTION OF THE DRAWINGS

The disclosure can be more fully understood by reading the subsequent detailed description and examples with references made to the accompanying drawings, wherein:

FIG. 1 is a schematic view showing a three-pathway system in one embodiment of the application, wherein the three-pathway system includes a pathway (110) of reacting ATP with luciferin, oxygen and luciferase to emit light (luminescence), a pathway (112) of reacting PPi derived from the pathway for luminescence (110) with ATP-sulfurylase and adenosine 5′ phosphosulfate (APS) to regenerate ATP, and a pathway (114) of reacting AMP derived from the pathway for luminescence (110) with uridine triphosphate (UTP) and ADK to form adenosine diphosphate (ADP) and reacting ADP with acetyl phosphate and acetate kinase to regenerate ATP;

FIG. 2 is a schematic view showing the luminescence produced by the two-pathway system and the three-pathway system according to Example 1;

FIG. 3 is a schematic view showing the luminescence according to Example 2, wherein the A column represents the luminescence from a pathway for luminescence only, the B column represents the luminescence from a two-pathway system containing a pathway for luminescence and a pathway for PPi reaction, the C column represents the luminescence from a two-pathway system containing a pathway for luminescence and a pathway for AMP reaction, and the D column represents the luminescence from a three-pathway system of one embodiment of the application;

FIG. 4 is a schematic view showing luminescence detection for the concentration of E. coli BL 21 by a two-pathway system and a three-pathway system;

FIG. 5 is a schematic view showing luminescence detection for the concentration of Psudomonas aeruginosa PAO1 by a two-pathway system and a three-pathway system; and

FIG. 6 is a schematic view showing luminescence detection for the concentration of B. cereus by a two-pathway system and a three-pathway system.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

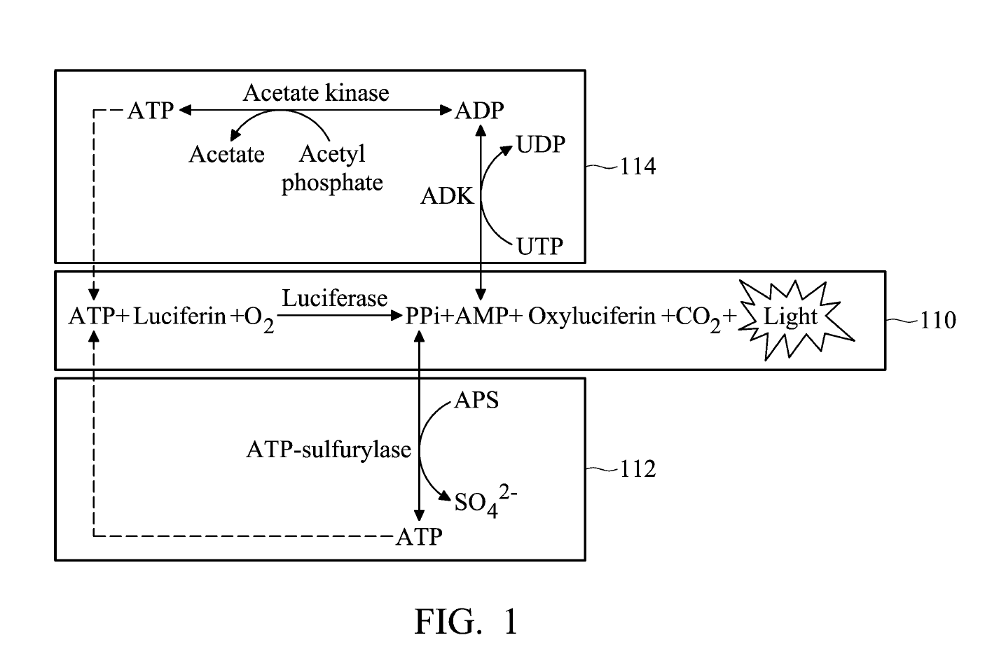

One embodiment of the application is a three-pathway system shown in FIG. 1. The pathway for luminescence (110) in FIG. 1 corresponds to the conventional luminescence detection, in which ATP reacts with luciferine and luciferase in the presence of oxygen gas to produce pyrophosphate (PPi), adenosine monophosphate (AMP), oxyluciferin and carbon dioxide (CO₂) and to emit light (luminescence). In the embodiment, the products of PPi and AMP are further used to regenerate ATP.

In the embodiment, the product, PPi, of the pathway for luminescence (110) reacts with adenosine triphosphate sulfurylase (ATP-sulfurylase) and adenosine 5′ phosphosulfate (APS) to regenerate ATP and produce sulfate ions (SO₄²⁻) in the pathway for PPi reaction (112) (shown in FIG. 1). Through the pathway for PPi reaction (112), the amount of ATP is increased and the regenerated ATP can be further cycled to the pathway for luminescence (110) to produce more light.

In the embodiment, the product, AMP, of the pathway for luminescence (110) reacts with adenylate kinase (ADK) and uridine triphosphate (UTP) to produce adenosine diphosphate (ADP), and the ADPs further reacts with acetate kinase and acetyl phosphate to produce ATP and acetate in the pathway for AMP reaction (114) (shown in FIG. 1). Similarly, through the pathway for AMP reaction (114), the amount of ATP is increased and the regenerated ATP can be further cycled to the pathway for luminescence (110) to produce more light.

According to the three-pathway system of one embodiment of the application, the concentration of ATP is able to be amplified several times to tens of times through repeating the three-pathway cycle, even if the initial concentration of ATP is very low. In one example, the concentration of ATP is amplified more than 5-fold by the three-pathway system when compared to that of the pathway for luminescence only.

Furthermore, when the regenerated ATP, obtained from either the pathway for PPi reaction (112) or the pathway for AMP reaction (114), repeats the pathway for luminescence (110), more light is produced and detectable. Therefore, detection sensitivity can be increased without altering conventional luminometers. Except for luminescence detection, one skilled in the art can appropriately design the three-pathway system for relevant clinical or industrial uses.

In addition, conventional luminescence detection uses ATP in microbial cells as an initial substrate to detect the luminescence from the conversion of ATP to light. When the amount of microorganisms in a sample is minor, the luminescence is relatively low. As such, the impurity in the sample easily leads to background noises and the sensitivity of detection is insufficient. Therefore, the sensitivity of conventional luminescence detection is only up to 10⁴ CFU/ml of microorganisms.

The method for amplifying ATP according to the application uses the three-pathway system to amplify the amount of ATP and produce more light for detection. Therefore, the detection for the concentration of microorganisms by using the method of the application can effectively lower interference from background noises and the sensitivity can reach to less than 10⁴ CFU/ml of microorganisms. The sensitivity and stability of the detection can be thereby increased. In one example, the sensitivity, by using the method of the application, is approximately 10² CFU/ml of microorganisms, which is nearly the detection limit of conventional luminometers. Therefore, even if a sample contains a trace amount of microorganisms, the detection by using the method of the application is still sensitive and stable because ATP is repeatedly amplified and more light is produced.

According to the method of the application, all substrates and enzymes can be added into a tube at the same time for reaction. The one-tube and one-step process simplifies the detection operation and decreases the potential for human error. Alternatively, the substrates and enzymes may be added at different times according to their nature. For instance, luciferin and luciferase are substances for luminescence. For precise detection, they may be added to the last.

Substrates and enzymes can react in an open space and under room temperature. The time and place for reaction is not limited. Reaction preferably lasts for 30 seconds to 10 minutes for obtaining optimal detection levels.

The concentration of substrates and enzymes used in the method and reagent according to the application can be appropriately adjusted given the different operational apparatuses, environmental conditions, and sample statuses, etc. In one example, the concentration of ATP-sulfurylase is about 5×10⁻⁴˜5×10⁻⁶ U, the concentration of APS is about 0.1˜10 nM, the concentration of ADK is about 0.5˜5 U, the concentration of UTP is about 0.01˜1 μM, the concentration of acetate kinase is about 0.5˜5 U, the concentration of acetyl phosphate is about 5˜50 μM, the concentration of luciferin is about 50˜400 μM, and the concentration of luciferase is about 0.5˜10 U, but are not limited thereto.

In one example, the substrates and enzymes are stored in a Tris buffer (tris(hydroxymethyl)aminomethane buffer). The Tris buffer is in a concentration of about 25˜75 mM, but is not limited thereto.

In one example, the buffer may further contain Mg²⁺ ions at a concentration of 2˜10 mM for luciferase activity. The Mg²⁺ ions may be present in the buffer by adding MgCl₂.

The method for detecting the concentration of microorganisms of the application can be applied to drinking water, domestic water, industrial water, biosamples or the likes.

Based on the three pathways of the disclosure, the application provides a novel method for amplifying ATP and for detecting the concentration of microorganisms with high sensitivity and stability with a simplified operation technique.

The examples below show luminescence comparisons by using the three-pathway system according to the application and the conventional luminescence detection (a pathway for luminescence), a two-pathway system containing a pathway for luminescence and a pathway for PPi reaction and a two-pathway system containing a pathway for luminescence and a pathway for AMP reaction. Note that the examples are for the purpose of further describing the effects of the application. Thus, the scope of the invention is not limited thereto, which should be based on the claims thereafter.

### Example 1

### Comparison of Luminescence Between a Two-Pathway System and a Three-Pathway System

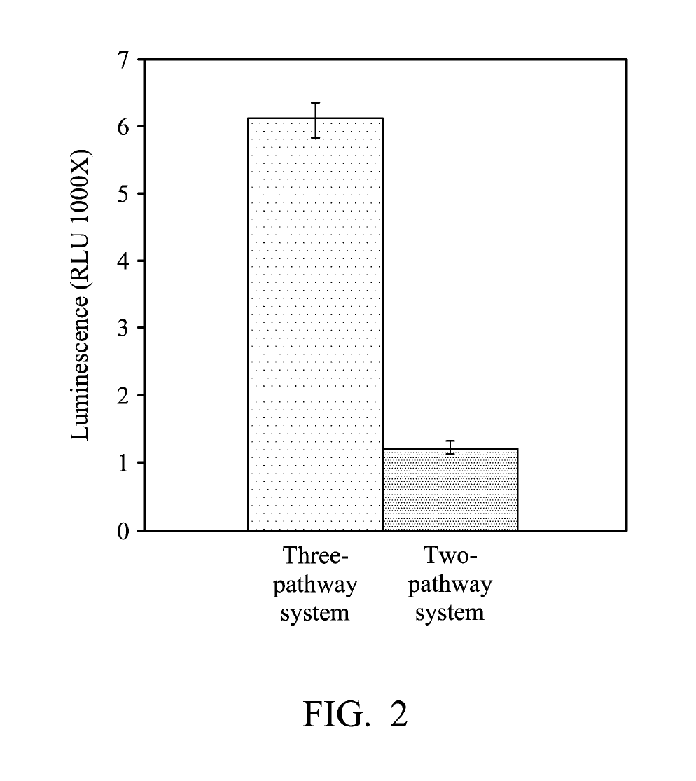

Substrates and enzymes were respectively added into a TD-20/20 luminometer (Turner Designs, Sunnyvale, Calif.) at concentrations as shown in Table 1. After 30 seconds, the luminescence (RLU 1000X) produced by the two-pathway and three-pathway systems was detected, respectively. The steps were repeated three times and an average luminescence was calculated. The results are shown in FIG. 2.

TABLE 1

| Reactants | | | Three-pathway | | Two-pathway | |
|---|---|---|---|---|---|---|
| (Substrates/enzymes) | concentration | | system | | system | |
| ATP-sulfurylase⁽¹⁾ | 5 × 10⁻⁵ | U | 5 | μl | 5 | μl |
| APS⁽²⁾ | 0.1 | μM | 5 | μl | 5 | μl |
| Acetyl phosphate(AcoP)⁽³⁾ | 10 | μM | 5 | μl | 5 | μl |
| PPi⁽⁴⁾ | 100 | pM | 5 | μl | 5 | μl |
| ADK⁽⁵⁾ | 1 | unit | 5 | μl | — | |
| Acetate kinase⁽⁶⁾ | 1 | unit | 5 | μl | — | |
| UTP⁽⁷⁾ | 20 | μM | 5 | μl | — | |
| Buffer 1⁽⁸⁾ | | | 10 | μl | 30 | μl |
| Buffer 2⁽⁹⁾ | | | 50 | μl | 50 | μl |
| Total volume | | | 100 | μl | 100 | μl |

⁽¹⁾ATP-sulfurylase is obtained from Sigma A8957-10 UN.
⁽²⁾APS is obtained from Sigma A5508-5 MG.
⁽³⁾Acetyl phosphate (AcoP) is obtained from sigma A0262-500 MG.
⁽⁴⁾PPi is obtained from 27 mg of PPi (Nacalai tesque 31816-25 500 G) in 1 ml ddH₂O.
⁽⁵⁾ADK is obtained from 100 UN of ADK (Sigma M3003 1 KU) in 100 μl of Tris solution. 1 μl represents 1 UN.
⁽⁶⁾Acetate kinase is obtained from Sigma A7437-250 UN.
⁽⁷⁾UTP is obtained from 5.5 mg of UDP (Sigma U6750 100 MG) in 1 ml ddH₂O.
⁽⁸⁾Buffer 1: 50 mM Tris buffer, pH 7.6.
⁽⁹⁾Buffer 2: 6 mM of MgCl₂, 1 U of luferase (Sigma L9506-1 MG) and 1mM of luferin (Sigma L6882-.2 MG) in 1 ml of Buffer 1.

As shown in FIG. 2, the luminescence through the three-pathway system has an average of 6065 RLU, while that through the two-pathway system has an average of 1158 RLU. The result exhibits that the three-pathway system produces about a 5-fold luminescence over that of the two-pathway system for a short reaction time, representing that the three-pathway system increases the sensitivity of detection by a luminometer.

### Example 2

### Comparison of Luminescence Between the Three-Pathway System, the Two-Pathway System and Conventional Luminescence Detection

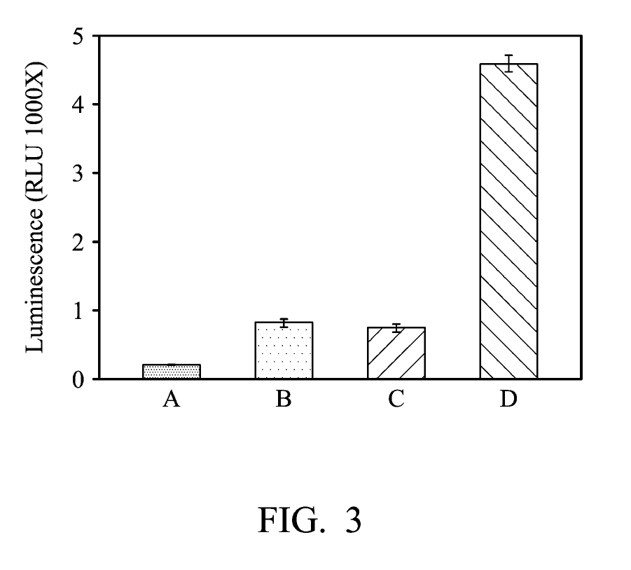

The reactants prepared in Example 1 were added to a TD-20/20 luminometer (Turner Designs, Sunnyvale, Calif.) at concentrations as shown in Table 2. After 3 minutes, the luminescence (RLU 1000X) produced by three-pathway system, the two-pathway system and conventional luminescence detection technique was detected, respectively. The steps were repeated three times and an average luminescence was calculated. The results are shown in FIG. 3 and Table 3.

TABLE 2
The reactants and concentrations with
respect to columns A~D in FIG. 3

| Reactants | Concentration | | Volume | |
|---|---|---|---|---|
| Group A | | | | |
| ATP⁽¹⁰⁾ | 1 | pM | 5 | μl |
| Buffer 1 | | | 45 | μl |
| Buffer 2 | | | 50 | μl |
| Total volume | | | 100 | μl |
| Group B | | | | |
| ATP-sulfurylase | 5 × 10⁻⁵ | U | 5 | μl |
| APS | 0.1 | μM | 5 | μl |
| ATP | 1 | pM | 5 | μl |
| Buffer 1 | | | 35 | μl |
| Buffer 2 | | | 50 | μl |
| Total volume | | | 100 | μl |
| Group C | | | | |
| Acetyl phosphate(AcoP) | 10 | μM | 5 | μl |
| ATP | 1 | pM | 5 | μl |
| ADK | 1 | unit | 5 | μl |
| Acetate kinase | 1 | unit | 5 | μl |
| UTP | 20 | μM | 5 | μl |
| Buffer 1 | | | 25 | μl |
| Buffer 2 | | | 50 | μl |
| Total volume | | | 100 | μl |
| Group D | | | | |
| ATP- sulfurylase | 5 × 10⁻⁵ | U | 5 | μl |
| APS | 0.1 | μM | 5 | μl |
| Acetyl acetate (AcoP) | 10 | μM | 5 | μl |
| ATP⁽¹⁰⁾ | 1 | pM | 5 | μl |
| ADK | 1 | unit | 5 | μl |
| Acetate kinase | 1 | unit | 5 | μl |
| UTP | 20 | μM | 5 | μl |
| Buffer 1 | | | 15 | μl |
| Buffer 2 | | | 50 | μl |
| Total volume | | | 100 | μl |

⁽¹⁰⁾ATP is obtained from 5.5 mg of ATP (Sigma A2383 25 G) in 1 ml ddH₂O.

TABLE 3

| Groups/Columns | Pathways | Luminescence (RLU) |
|---|---|---|
| A | One pathway for luminescence | 209.1 |
| B | Two-pathway system containing a | 808.4 |
| | pathway for luminescence and | |
| | a pathway for PPi reaction | |
| C | Two-pathway system containing a | 717.5 |
| | pathway for luminescence and | |
| | a pathway for AMP reaction | |
| D | Three-pathway system | 4521.7 |

As shown in Table 3 and FIG. 3, the three-pathway system (column D) produces more than 20-fold the luminescence when compared to that of conventional luminescence detection techniques (column A). Compared to the two-pathway system (columns B and C), the luminescence from the three-pathway system (D column) increased 6-fold.

### Example 3

### Two-Pathway System and Three-Pathway System on Different Strains

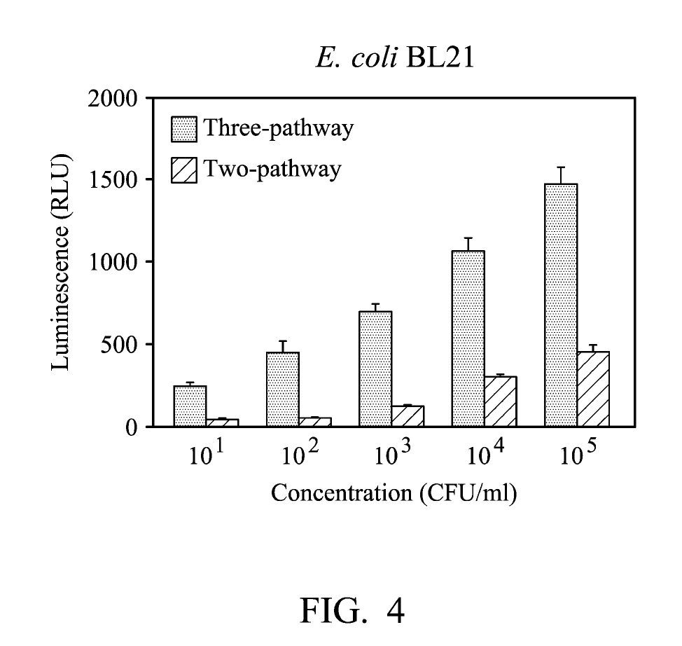

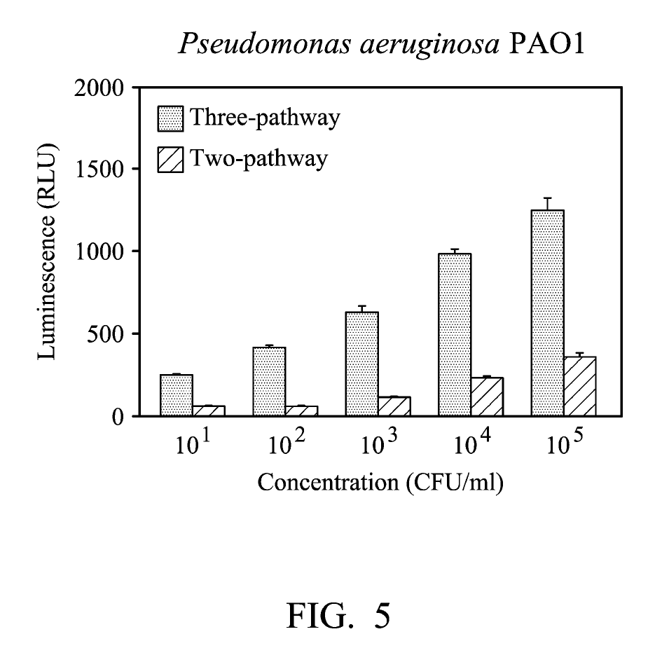

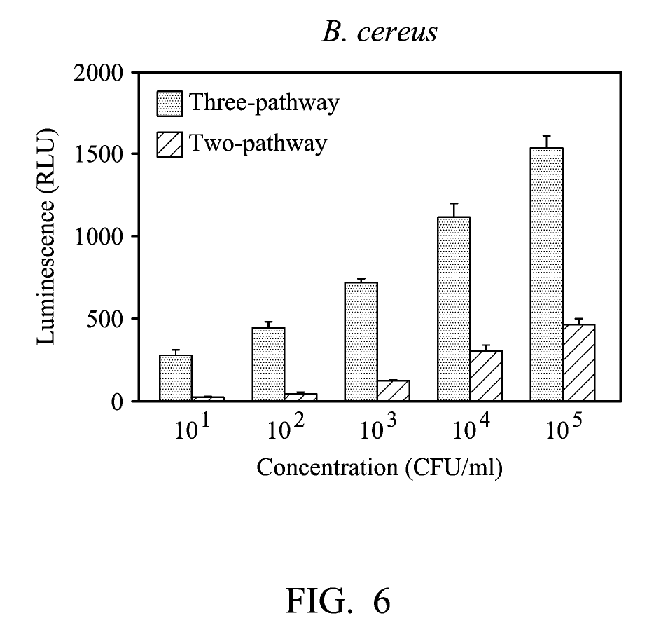

The strains, E. coli BL21, Psudomonas aeruginosa PAO1 and B. cereus, were used for detection. Each strain was incubated in an LB medium at a concentration of 10¹˜10⁵ CFU/ml and separated into two groups. One group was detected by the three-pathway system, and the other group was detected by a two-pathway system containing a pathway for luminescence and a pathway for PPi reaction. The reaction conditions were as Example 1. The results are shown in FIGS. 4, 5 and 6, respectively.

According to the results, detection of microorganisms at a low concentration (such as 10¹ or 10² CFU/ml) by using the two-pathway system shows weak luminescence which does not effectively identify the presence of microorganisms by the luminometer. However, by using the three-pathway system according to the application, the luminescence obtained from the microorganisms at a concentration of less than 10² CFU/ml was within the detectable range of luminometers. Therefore, detection by the three-pathway system can efficiently determine the concentration of microorganisms in a sample. In other words, the method according to the application is capable of identifying a trace amount of microorganisms in a sample, such that detection sensitivity is increased to over that of conventional techniques.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for amplifying adenosine triphosphate (ATP), comprising:
mixing adenosine triphosphate sulfurylase (ATP-sulfurylase), adenosine 5′ phosphosulfate (APS), adenylate kinase (ADK), uridine triphosphate (UTP), acetate kinase, acetyl phosphate, luciferin and luciferase in the presence of ATP to form a mixture, and
reacting the mixture to amplify ATP.

2. The method as claimed in claim 1, wherein the concentration of the adenosine triphosphate sulfurylase is 5×10⁻⁴˜5×10⁻⁶ U, the concentration of the adenosine 5′ phosphosulfate is 0.1˜10 nM, the concentration of the adenylate kinase is 0.5˜5 U, the concentration of the uridine triphosphate is 0.01˜1 μM, the concentration of the acetate kinase is 0.5˜5 U, the concentration of the acetyl phosphate is 5˜50 μM, the concentration of the luciferin is 50˜400 μM, and the concentration of the luciferase is 0.5˜10 U.

3. The method as claimed in claim 1, wherein the reacting step is a one-step process.

4. A method for detecting the concentration of microorganisms in a sample, comprising:
mixing the sample with adenosine triphosphate sulfurylase (ATP-sulfurylase), adenosine 5′ phosphosulfate (APS), adenylate kinase (ADK), uridine triphosphate (UTP), acetate kinase, acetyl phosphate, luciferin and luciferase to form a mixture;
reacting the mixture to obtain luminescence, and
determining the concentration of microorganisms according to the luminescence.

5. The method as claimed in claim 4, wherein the sample comprises drinking water, domestic water, industrial water or biosamples.

6. The method as claimed in claim 4, wherein the concentration of the adenosine triphosphate sulfurylase is 5×10⁻⁴˜5×10⁻⁶ U, the concentration of the adenosine 5′ phosphosulfate is 0.1˜10 nM, the concentration of the adenylate kinase is 0.5˜5 U, the concentration of the uridine triphosphate is 0.01˜1 μM, the concentration of the acetate kinase is 0.5˜5 U, the concentration of the acetyl phosphate is 5˜50 μM, the concentration of the luciferin is 50˜400 μM, and the concentration of the luciferase is 0.5˜10 U.

7. The method as claimed in claim 4, wherein the reacting step is a one-step process.

8. The method as claimed in claim 4, for detecting microorganisms at a concentration of less than 10⁴ CFU/ml.

9. A reagent for detecting a concentration of microorganisms, comprising adenosine triphosphate sulfurylase (ATP-sulfurylase), adenosine 5′ phosphosulfate (APS), adenylate kinase (ADK), uridine triphosphate (UTP), acetate kinase, acetyl phosphate, luciferin and luciferase.

10. The reagent as claimed in claim 9, wherein the concentration of the adenosine triphosphate sulfurylase is 5×10⁻⁴˜5×10⁻⁶ U, the concentration of the adenosine 5′ phosphosulfate is 0.1˜10 nM, the concentration of the adenylate kinase is 0.5˜5 U, the concentration of the uridine triphosphate is 0.01˜1 μM, the concentration of the acetate kinase is 0.5˜5 U, the concentration of the acetyl phosphate is 5˜50 μM, the concentration of the luciferin is 50˜400 μM, and the concentration of the luciferase is 0.5˜10 U.

11. The reagent as claimed in claim 9, for detecting the concentration of microorganisms in drinking water, domestic water, industrial water or biosamples.

12. The reagent as claimed in claim 9, for detecting the microorganisms at a concentration less than 10⁴ CFU/ml.
